(19) United States
(12) Patent Application Publication
(10) Pub. No.: US 2019/0331686 A1
(43) Pub. Date: Oct. 31, 2019

(54) Methods and Systems for Determining the Risk of Developing Ovarian Cancer

(71) Applicant: Laboratory Corporation of America Holdings, Burlington, NC (US)

(72) Inventors: Stanley Ian Letovsky, Milton, MA (US); Russell Philip Grant, Chapel Hill, NC (US); Charles Donald Kafader, II, Johnstown, OH (US); Christopher Michael Shuford, Mebane, NC (US)

(21) Appl. No.: 16/394,517

(22) Filed: Apr. 25, 2019

Related U.S. Application Data
(60) Provisional application No. 62/663,640, filed on Apr. 27, 2018.

(51) Int. Cl. G01N 33/574

(52) U.S. Cl. CPC G01N 33/57449; C12Q 1/6886; G01N 33/57484

(57) ABSTRACT

The present invention relates to systems and methods for utilizing the measurement of two or more biomarkers to determine a probabilistic assessment for developing ovarian cancer. Particularly, aspects of the present invention are directed to a computer implemented method that includes obtaining, by a computing device, measured levels of two or more biomarkers in a sample obtained from a subject without knowledge of an ovarian mass or tumor in the subject, and determining, by the computing device, a probabilistic assessment of the subject developing ovarian cancer based on the obtained values.

### CROSS-REFERENCE TO RELATED APPLICATION

This application claims priority to U.S. Provisional Application No. 62/663,640, filed on Apr. 27, 2018, the entirety of which is incorporated herein by reference.

### FIELD OF THE INVENTION

The present invention relates to risk assessment of ovarian cancer, and in particular to systems and methods for utilizing the measurement of two or more biomarkers to determine a probabilistic assessment for developing ovarian cancer.

### BACKGROUND

Ovarian cancer is a growth of abnormal malignant cells that begins in the ovaries (women's reproductive glands that produce eggs), fallopian tubes (and then migrates to the ovaries), or the peritoneum. Ovarian tumors can be benign (noncancerous) or malignant (cancerous). Although abnormal, cells of benign tumors do not metastasize (spread to other parts of the body). Malignant cancer cells in the ovaries can metastasize in two ways: directly to other organs in the pelvis and abdomen (the more common way); or through the bloodstream or lymph nodes to other parts of the body.

Timely diagnosis of ovarian cancer has been elusive (e.g., about 20% of ovarian cancers are found at an early stage). Early-stage ovarian cancer rarely causes any symptoms. Advanced-stage ovarian cancer may cause few and nonspecific symptoms that are often mistaken for more common benign conditions. Thus, ovarian cancer often goes undetected until it has metastasize. At this late stage, ovarian cancer is more difficult to treat and is frequently fatal. Early-stage ovarian cancer, in which the disease is confined to the ovary, is more likely to be treated successfully. For example, when ovarian cancer is found early at a localized stage, about 94% of patients live longer than 5 years after diagnosis. Consequently, there has been a lot of research to develop a screening test for ovarian cancer, but there has not been much success so far. Conventional screening techniques include a pelvic exam and a transvaginal ultrasound (TVUS). TVUS is a test that uses sound waves to look at the uterus, fallopian tubes, and ovaries by putting an ultrasound wand into the vagina. TVUS can help find a mass (tumor) in the ovary, but TVUS cannot actually tell if a mass is cancer or benign. When TVUS is used for screening, most of the masses found are not cancer.

In order to improve upon conventional techniques to screen for ovarian cancer, a primary goal for researchers has been to optimize screening using molecular biomarkers. Biomarkers for risk assessment of cancer are useful for early detection and prevention of cancer, and have the potential for use in large-scale screening protocols. The most often used biomarker to screen for ovarian cancer is protein Cancer Antigen (CA) 125. In many women with ovarian cancer, levels of CA-125 are high. Thus, this test can be useful as a biomarker to help guide treatment in women known to have ovarian cancer, because a high level of CA-125 often goes down if treatment is working. However, checking CA-125 levels has not been found to be optimal as a screening test for ovarian cancer. The problem with using this test for screening is that common conditions other than cancer can also cause high levels of CA-125. In women who have not been diagnosed with cancer, a high CA-125 level is more often caused by one of these other conditions and not ovarian cancer. In addition, not everyone who has ovarian cancer has a high CA-125 level. Accordingly, the need exists for improved systems and methods for risk assessment of ovarian cancer.

### BRIEF SUMMARY

In various embodiments, a system is provided comprising: a logistic regression model that uses an equation comprising: input values comprising measured levels of two or more biomarkers in a sample obtained from a subject without knowledge of an ovarian mass or tumor in the subject, where the two or more biomarkers include Human epididymal protein 4 (HE4) and Cancer Antigen 125 (CA-125); coefficient values that take into consideration a time to diagnosis variable, where the input values are combined linearly using the coefficient values to predict an output value, and the output value is a differentiator between subjects that will develop ovarian cancer and subjects that will not develop ovarian cancer; one or more processors and non-transitory machine readable storage medium; and program instructions to determine a probabilistic assessment of the subject developing an ovarian carcinoma based at least on the logistic regression model. The program instructions are stored on the non-transitory machine readable storage medium for execution by the one or more processors.

In some embodiments, the time to diagnosis variable is estimated from training data and indicates a time from when biological samples having levels of the two or more biomarkers were obtained from test subjects to a time of the test subjects, respectively, being diagnosed with the ovarian carcinoma. Optionally, the determining the probabilistic assessment comprises executing the equation using the measured levels of the two or more biomarkers and the coefficient values. Optionally, the input values further comprise risk factors or factors that lower the risk of the ovarian carcinoma. Optionally, the risk factors include at least one of the following: age of the subject, family history, genetic mutation, inherited genetic disorder, and prior cancer. Optionally, the factors that lower the risk of the ovarian carcinoma include at least one of the following: child bearing status, use of birth control, use of oral contraceptives, and prior gynecological surgery. Optionally, the two or more biomarkers further include one or more biomarkers that has not been demonstrated previously to be predictive for the development ovarian cancer.

In various embodiments, a non-transitory machine readable storage medium is provided having instructions stored thereon that when executed by one or more processors cause the one or more processors to perform a method. The method comprising: selecting a logistic regression model, wherein the logistic regression model uses an equation comprising: (i) input values comprising measured levels of two or more biomarkers in a sample obtained from a subject without knowledge of an ovarian mass or tumor in the subject; and (ii) coefficient values estimated from training data that take into consideration various unspecified omitted factors, where the input values are combined linearly using the coefficient values to predict an output value, and the output value is a differentiator between subjects that will develop ovarian cancer and subjects that will not develop ovarian cancer; and determining a probabilistic assessment of the subject developing ovarian cancer based at least on the logistic regression model.

In some embodiments, the two or more biomarkers include two or more biomarkers that have not been demonstrated previously to be predictive for the development of ovarian cancer. Optionally, the two or more biomarkers include any combination of: Human epididymal protein 4 (HE4), Cancer Antigen 125 (CA-125), Leptin, Osteopontin (OPN), Prolactin, and Insulin-like Growth Factor 2 (IGF2). Optionally, the equation further comprise: (iii) coefficient values estimated from training data that take into consideration a time to diagnosis variable, wherein the time to diagnosis variable indicates a time from when biological samples having levels of the two or more biomarkers were obtained from test subjects to a time of the test subjects, respectively, being diagnosed with the ovarian carcinoma. Optionally, the determining the probabilistic assessment comprises executing the equation using the measured levels of the two or more biomarkers and the coefficient values. Optionally, the input values further comprise risk factors or factors that lower the risk of the ovarian carcinoma. Optionally, the method further comprises storing the probabilistic assessment.

In various embodiments, a method is provided comprising: selecting, using a computing device, a logistic regression model, wherein the logistic regression model uses an equation comprising: (i) input values comprising measured levels of two or more biomarkers in a sample obtained from a subject without knowledge of an ovarian mass or tumor in the subject; and (ii) coefficient values estimated from training data that take into consideration various unspecified omitted factors, where the input values are combined linearly using the coefficient values to predict an output value, and the output value is a differentiator between subjects that will develop ovarian cancer and subjects that will not develop ovarian cancer; and determining, using the computing device, a probabilistic assessment of the subject developing ovarian cancer based at least on the logistic regression model.

In some embodiments, the two or more biomarkers include two or more biomarkers that have not been demonstrated previously to be predictive for the development of ovarian cancer. Optionally, the two or more biomarkers include any combination of: Human epididymal protein 4 (HE4), Cancer Antigen 125 (CA-125), Leptin, Osteopontin (OPN), Prolactin, and Insulin-like Growth Factor 2 (IGF2). Optionally, the equation further comprise: (iii) coefficient values estimated from training data that take into consideration a time to diagnosis variable, wherein the time to diagnosis variable indicates a time from when biological samples having levels of the two or more biomarkers were obtained from test subjects to a time of the test subjects, respectively, being diagnosed with the ovarian carcinoma. Optionally, the determining the probabilistic assessment comprises executing the equation using the measured levels of the two or more biomarkers and the coefficient values. Optionally, the input values further comprise risk factors or factors that lower the risk of the ovarian carcinoma.

In various embodiments, a method is provided for assessing risk of developing ovarian cancer. The method comprises obtaining, by a computing device, measured levels of two or more biomarkers in a sample obtained from a subject without knowledge of an ovarian mass or tumor in the subject, where the two or more biomarkers include Human epididymal protein 4 (HE4) and Cancer Antigen 125 (CA-125); determining, by the computing device, a probabilistic assessment of the subject developing ovarian cancer based at least on the obtained values and coefficient values estimated from training data that take into consideration a time to diagnosis variable; and storing, by the computing device, the probabilistic assessment.

In some embodiments, the method further comprises selecting, by the computing device, one or more classifiers or logistic regression equations for assessing the risk of the subject developing ovarian cancer. Optionally, the determining the probabilistic assessment comprises executing the one or more classifiers or logistic regression equations using the measured levels of the two or more biomarkers and the coefficient values. Optionally, the method further comprises obtaining, by the computing system, one or more risk factors or factors that lower the risk of the ovarian carcinoma. Optionally, the one or more classifiers or logistic regression equations are selected based on at least one of: (i) the two or more biomarkers, and (ii) the risk factors or factors that lower the risk of the ovarian carcinoma. Optionally, the determining the probabilistic assessment comprises executing the one or more classifiers or logistic regression equations using the measured levels of the two or more biomarkers and the one or more risk factors or factors that lower the risk of the ovarian carcinoma. Optionally, the method further comprises providing, by the computing system, a recommended frequency of follow-up testing for the two or more biomarkers based on the one or more classifiers or logistic regression equations selected to determine the probabilistic assessment of the subject.

In various embodiments, a non-transitory machine readable storage medium is provided having instructions stored thereon that when executed by one or more processors cause the one or more processors to perform a method. The method comprising obtaining measured levels of two or more biomarkers in a sample obtained from a subject without knowledge of an ovarian mass or tumor in the subject, wherein the two or more biomarkers include Human epididymal protein 4 (HE4) and Cancer Antigen 125 (CA-125); determining a probabilistic assessment of the subject developing ovarian cancer based at least on the obtained values and coefficient values estimated from training data that take into consideration a time to diagnosis variable; and storing the probabilistic assessment.

In some embodiments, the method further comprises selecting one or more classifiers or logistic regression equations for assessing the risk of the subject developing ovarian cancer. Optionally, the determining the probabilistic assessment comprises executing the one or more classifiers or logistic regression equations using the measured levels of the two or more biomarkers and the coefficient values. Optionally, the method further comprises obtaining one or more risk factors or factors that lower the risk of the ovarian carcinoma. Optionally, the one or more classifiers or logistic regression equations are selected based on at least one of: (i) the two or more biomarkers, and (ii) the risk factors or factors that lower the risk of the ovarian carcinoma. Optionally, the determining the probabilistic assessment comprises executing the one or more classifiers or logistic regression equations using the measured levels of the two or more biomarkers and the one or more risk factors or factors that lower the risk of the ovarian carcinoma. Optionally, the method further comprises providing a recommended frequency of follow-up testing for the two or more biomarkers based on the one or more classifiers or logistic regression equations selected to determine the probabilistic assessment of the subject.

In various embodiments, a system is provided comprising: one or more processors and non-transitory machine readable storage medium; program instructions to obtain, by a computing device, measured levels of two or more biomarkers in a sample obtained from a subject without knowledge of an ovarian mass or tumor in the subject, wherein the two or more biomarkers include Human epididymal protein 4 (HE4) and Cancer Antigen 125 (CA-125); program instructions to determine, by the computing device, a probabilistic assessment of the subject developing ovarian cancer based at least on the obtained values and coefficient values estimated from training data that take into consideration a time to diagnosis variable; and program instructions to store, by the computing device, the probabilistic assessment. The program instructions are stored on the non-transitory machine readable storage medium for execution by the one or more processors.

In some embodiments, the system further comprises program instructions to select one or more classifiers or logistic regression equations for assessing the risk of the subject developing ovarian cancer. Optionally, the determining the probabilistic assessment comprises executing the one or more classifiers or logistic regression equations using the measured levels of the two or more biomarkers and the coefficient values. Optionally, the system further comprises program instructions to obtain one or more risk factors or factors that lower the risk of the ovarian carcinoma. Optionally, the one or more classifiers or logistic regression equations are selected based on at least one of: (i) the two or more biomarkers, and (ii) the risk factors or factors that lower the risk of the ovarian carcinoma. Optionally, the determining the probabilistic assessment comprises executing the one or more classifiers or logistic regression equations using the measured levels of the two or more biomarkers and the one or more risk factors or factors that lower the risk of the ovarian carcinoma. Optionally, the system further comprises program instructions to provide a recommended frequency of follow-up testing for the two or more biomarkers based on the one or more classifiers or logistic regression equations selected to determine the probabilistic assessment of the subject.

### BRIEF DESCRIPTION OF THE DRAWINGS

The present invention will be better understood in view of the following non-limiting figures, in which:

FIG. 1 shows an illustrative architecture of a computing system implemented in accordance with various embodiments;

FIG. 2 shows an exemplary flow for assessing an ovarian cancer risk in accordance with various embodiments;

FIG. 3 shows Receiver Operating Characteristic (ROC) curves for the logistic regression analysis of each of the six biomarkers in accordance with various embodiments;

FIG. 4 shows an area under the ROC curves (AUC) being used to evaluate test accuracy in accordance with various embodiments;

FIGS. 5A-5F show differentiation between cases and controls in accordance with various embodiments;

FIG. 6 shows accumulated data from the cases and controls that demonstrates a link between sensitivity/specificity of the logistic regression analysis and the time at which the sample was obtain prior to diagnosis in accordance with various embodiments;

FIG. 7 shows a decision tree algorithm generated in accordance with various embodiments;

FIG. 8 shows an AUC of the ROC for an exemplary logistic regression equation in accordance with various embodiments;

FIG. 9 shows data from the AUC of the ROC for an exemplary logistic regression equation in accordance with various embodiments;

FIG. 10 shows an exemplary two variable model for 1.0 years in accordance with various embodiments;

FIG. 11 shows an exemplary two variable model for 1.5 years in accordance with various embodiments;

FIG. 12 shows an AUC of the ROC for an exemplary logistic regression equation in accordance with various embodiments; and

FIGS. 13A, 13B, and 13C show cross validated time-thresholded AUCs for different models used to generate classifiers or logistic regression equations in accordance with various embodiments.

### DETAILED DESCRIPTION

### I. Introduction

In various embodiments, techniques are provided for determining a risk of a subject (e.g., a human subject) to develop an ovarian carcinoma without knowledge of an ovarian mass or tumor (e.g., a preexisting ovarian mass or tumor) in the subject. In certain embodiments, the subject has one or more symptoms and/or risk factors for an ovarian carcinoma. The symptoms of ovarian carcinoma may include without limitation abdominal bloating, indigestion or nausea; changes in appetite, such as a loss of appetite or feeling full sooner; pressure in the pelvis or lower back; more frequent or urgent need to urinate and/or constipation; changes in bowel movements; increased abdominal girth; tiredness or low energy; and changes in menstruation. The risk factors of ovarian carcinoma may include without limitation: age greater than 55 of the woman; a family history such as women with a mother, sister, grandmother or aunt who has had ovarian cancer have a higher risk of developing the disease; genetic mutation such as women with the BRCA1 mutation have a 35 to 70 percent higher risk of ovarian cancer, and women with the BRCA2 mutation have a 10 to 30 percent higher risk; an inherited genetic disorder such as lynch syndrome or peutz-jeghers syndrome; and prior cancer such as breast, colorectal, or endometrial cancer.

The systems and methods of the various embodiments described herein may utilize hardware and/or software (e.g., program instructions executed by one or more processors) to determine levels of two or more biomarkers in a biological sample of the subject, and, according to the levels, determine a probabilistic assessment of the subject developing an ovarian carcinoma. As used herein, the term “biological sample” refers to a biological specimen including, for example, blood, tissue, urine, etc. taken from a participant. In some embodiments, the biological specimen is whole blood, serum, or plasma. As used herein, the term “participant” or “subject” refers to a human that has not been diagnosed with an ovarian mass or tumor. In other words, the participant or subject does not have an existing or present-day ovarian tumor (e.g., prior to Stage I ovarian cancer or prior to recurrence). As used herein, the term “mass” or “tumor” refers to groups of abnormal cells that form lumps or growths in the subject. The mass or tumor may be benign (not cancerous), premalignant (not yet cancerous but may develop into a cancerous tumor), or malignant (cancerous).

Conventional approaches for screening of ovarian cancer include contacting a biological sample from a subject with at least one antibody specific for a biomarker (e.g., CA-125) to determine the presence of the biomarker in the biological sample, and therefrom detecting the presence of an ovarian carcinoma. Unfortunately, elevated levels of biomarkers such as CA-125 alone or in combination with other known indicators, do not provide a definitive diagnosis of malignancy, or of a particular malignancy such as ovarian carcinoma. Therefore, additional biomarkers (e.g., human epididymis protein 4a (HE4a) polypeptides) that are overexpressed in certain malignancies have been proposed to screen for ovarian cancer. Both these approaches, however, are limited to screening for the presence of an ovarian carcinoma in a subject that has presently developed an ovarian mass or tumor, and have not been shown to be useful in assessing the risk of a subject prior to developing an ovarian mass or tumor such as an ovarian carcinoma. In other words, these approaches are limited to identification of a present ovarian carcinoma in a subject (i.e., a diagnostic), and have not been shown to be predictive in nature for risk assessment of developing an ovarian carcinoma.

To address these problems, the present invention is directed to systems and methods for utilizing the measurement of two or more biomarkers to determine a probabilistic assessment for developing ovarian cancer. For example, one illustrative embodiment of the present disclosure is directed to a computer implemented method that includes obtaining, by a computing device, measured levels of two or more biomarkers in a sample obtained from a subject without knowledge of an ovarian mass or tumor (e.g., a preexisting ovarian mass or tumor) in the subject, determining, by the computing device, a probabilistic assessment of the subject developing ovarian cancer based on the obtained values, and storing, by the computing device, the probabilistic assessment. In some embodiments, the method further includes selecting, by the computing device, one or more classifiers or logistic regression equations for assessing the risk of the subject developing ovarian cancer, and the determining the probabilistic assessment comprises executing the one or more classifiers or logistic regression equations using the measured levels of the two or more biomarkers.

### II. System Environment

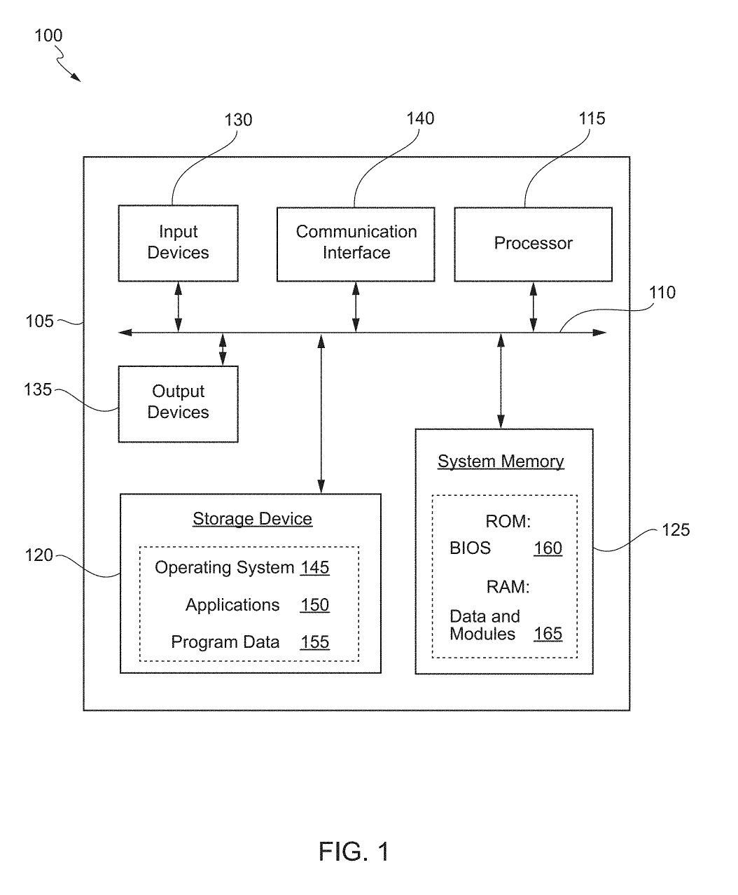

FIG. 1 is an illustrative architecture of a computing system 100 implemented in various embodiments. The computing system 100 is only one example of a suitable computing system and is not intended to suggest any limitation as to the scope of use or functionality of the present invention. Also, computing system 100 should not be interpreted as having any dependency or requirement relating to any one or combination of components illustrated in computing system 100. As shown in FIG. 1, computing system 100 includes a computing device 105. The computing device 105 can be resident on a network infrastructure such as within a cloud environment, or may be a separate independent computing device (e.g., a computing device of a service provider). The computing device 105 may include a bus 110, processor 115, a storage device 120, a system memory (hardware device) 125, one or more input devices 130, one or more output devices 135, and a communication interface 140.

The bus 110 permits communication among the components of computing device 105. For example, bus 110 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 105.

The processor 115 may be one or more conventional processors, microprocessors, or specialized dedicated processors that include processing circuitry operative to interpret and execute computer readable program instructions, such as program instructions for controlling the operation and performance of one or more of the various other components of computing device 105 for implementing the functionality, steps, and/or performance of the present invention. In certain embodiments, processor 115 interprets and executes the processes, steps, functions, and/or operations of the present invention, which may be operatively implemented by the computer readable program instructions. For example, processor 115 can select one or more biomarkers of interest and assess the value of the two or more biomarkers as a predictive or diagnostic tool for an ovarian carcinoma. The processor 115 can further obtain biological sample values for the two or more biomarkers in an individual, and determine a probabilistic assessment of the subject developing an ovarian carcinoma based on the obtained biological sample values. In embodiments, the selected two or more biomarkers, the obtained biological sample values, and the probabilistic assessment developed by the processor 115 can be stored in the storage device 120.

The storage device 120 may include removable/non-removable, volatile/non-volatile computer readable media, such as, but not limited to, non-transitory machine readable storage medium such as magnetic and/or optical recording media and their corresponding drives. The drives and their associated computer readable media provide for storage of computer readable program instructions, data structures, program modules and other data for operation of computing device 105 in accordance with the different aspects of the present invention. In embodiments, storage device 120 may store operating system 145, application programs 150, and program data 155 in accordance with aspects of the present invention.

The system memory 125 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 160 (BIOS) including the basic routines that help to transfer information between the various other components of computing device 105, such as during start-up, may be stored in the ROM. Additionally, data and/or program modules 165, such as at least a portion of operating system 145, program modules, application programs 150, and/or program data 155, that are accessible to and/or presently being operated on by processor 115, may be contained in the RAM. In embodiments, the program modules 165 and/or application programs 150 can comprise a user interface, a binary classifier system, the algorithms such as a decision tree and logistic regression, a comparison tool, and one or more databases, for example, of two or more biomarkers and the results of assays for such biomarkers in various biological samples, which provides the instructions and data for execution of the processor 115.

The one or more input devices 130 may include one or more mechanisms that permit an operator to input information to computing device 105, such as, but not limited to, a touch pad, dial, click wheel, scroll wheel, touch screen, one or more buttons (e.g., a keyboard), mouse, game controller, track ball, microphone, camera, proximity sensor, light detector, motion sensors, biometric sensor, and combinations thereof. The one or more output devices 135 may include one or more mechanisms that output information to an operator, such as, but not limited to, audio speakers, headphones, audio line-outs, visual displays, antennas, infrared ports, tactile feedback, printers, or combinations thereof.

The communication interface 140 may include any transceiver-like mechanism (e.g., a network interface, a network adapter, a modem, or combinations thereof) that enables computing device 105 to communicate with remote devices or systems, such as a mobile device or other computing devices such as, for example, a server in a networked environment, e.g., cloud environment. For example, computing device 105 may be connected to remote devices or systems via one or more local area networks (LAN) and/or one or more wide area networks (WAN) using communication interface 140.

As discussed herein, computing system 100 may be configured to determine a probabilistic assessment for developing ovarian cancer. In particular, computing device 105 may perform tasks (e.g., process, steps, methods and/or functionality) in response to processor 115 executing program instructions contained in non-transitory machine readable storage medium, such as system memory 125. The program instructions may be read into system memory 125 from another computer readable medium (e.g., non-transitory machine readable storage medium), such as data storage device 120, or from another device via the communication interface 140 or server within or outside of a cloud environment. In embodiments, an operator may interact with computing device 105 via the one or more input devices 130 and/or the one or more output devices 135 to facilitate performance of the tasks and/or realize the end results of such tasks in accordance with aspects of the present invention. In additional or alternative embodiments, hardwired circuitry may be used in place of or in combination with the program instructions to implement the tasks, e.g., steps, methods and/or functionality, consistent with the different aspects of the present invention. Thus, the steps, methods and/or functionality disclosed herein can be implemented in any combination of hardware circuitry and software.

### III. Risk Assessment Using Mathematical Models

In various embodiments, techniques are provided for determining a risk of a subject (e.g., a human subject) to develop an ovarian carcinoma without knowledge of an ovarian mass or tumor (e.g., a preexisting ovarian mass or tumor) in the subject. The techniques may utilize the computing system 100 described with respect to FIG. 1 to determine levels of two or more biomarkers in a biological sample of the subject, and, according to the levels, determine a probabilistic assessment of the subject developing an ovarian carcinoma. In some embodiments, the biomarkers and biomarker combinations are to be measured in blood samples, e.g., serum, from various subjects that have not developed an ovarian mass or tumor (diagnosed or undiagnosed). Examples of subjects from which such a sample may be obtained and utilized in accordance with various embodiments discussed herein include, but are not limited to, asymptomatic subjects that have not developed a ovarian mass or tumor, subjects manifesting or exhibiting one or more symptoms of ovarian cancer that have not developed an ovarian mass or tumor, subjects predisposed to ovarian cancer (e.g., subjects with a family history of ovarian cancer, subjects with a genetic predisposition to ovarian cancer, subjects suspected of having ovarian cancer, subjects not undergoing treatment for ovarian cancer and have not developed an ovarian mass or tumor, subjects determined by a medical practitioner (e.g., a physician) to be healthy or ovarian cancer-free (i.e., normal), subjects that have been cured of ovarian cancer and that have not developed recurrence of an ovarian mass or tumor, subjects that have not been diagnosed with ovarian cancer and it is unknown as to whether they developed an ovarian mass or tumor, and subjects that have not been diagnosed with ovarian cancer and have not developed an ovarian mass or tumor.

In various embodiments, biomarkers and biomarker combinations are selected from known biomarkers that have been shown to have some diagnostic utility for ovarian cancer. For example, a measurement of the level of the biomarkers is predictive for the development ovarian cancer. In some embodiments, biomarkers and biomarker combinations are identified or determined as potential biomarkers of ovarian cancer. For example, a measurement of the level of the biomarkers has not been demonstrated previously to be predictive for the development of ovarian cancer. In other embodiments, biomarkers and biomarker combinations are selected from biomarkers that are autonomously identified as potential biomarkers of ovarian cancer from databases of data, e.g., data mined from medical publications. For example, a measurement of the level of the biomarkers has not been demonstrated previously to be predictive for the development of ovarian cancer. In some embodiments, the biomarkers and combination of biomarkers include any combination of human epididymal protein 4 (HE4), Cancer Antigen 125 (CA-125), Leptin, Osteopontin (OPN), Prolactin, and Insulin-like Growth Factor 2 (IGF2). In certain embodiments, the biomarkers are HE4 and CA-125. In other embodiments, the biomarkers and combination of biomarkers include any combination of: HE4, CA-125, and one or more other biomarkers that has not been demonstrated previously to be predictive for the development of ovarian cancer. In yet other embodiments, the biomarkers and combination of biomarkers include any combination of two or more biomarkers that have not been demonstrated previously to be predictive for the development of ovarian cancer.

In order to determine a risk of a subject to develop ovarian cancer a mathematical model may be used to generate “classifiers” which differentiate between subjects that will develop ovarian cancer and subjects that will not develop ovarian cancer. Mathematical models useful in accordance with various embodiments include those using either supervised or unsupervised learning. In some embodiments, the mathematical model chosen uses supervised learning in conjunction with a “training population” to evaluate each of the possible combination of biomarkers. In some embodiments, the mathematical model used is selected from the following: a regression model, a logistic regression model, a neural network, a clustering model, principal component analysis, nearest neighbor classifier analysis, linear discriminant analysis, quadratic discriminant analysis, a support vector machine, a decision tree, a genetic algorithm, classifier optimization using bagging, classifier optimization using boosting, classifier optimization using the Random Subspace Method, a projection pursuit, and weighted voting. In certain embodiments, a logistic regression model is used. In another embodiment, a neural network model is used. In yet another embodiment, mathematical models can be used in combination.

Populations used for training input into the mathematical model should be chosen so as to result in statistically significant resulting biomarker combinations. In some embodiments, the reference or training population includes between 10 and 30 subjects. In another embodiment, the training population contains between 30 and 50 subjects. In still other embodiments, the reference population includes two or more populations each containing between 50 and 100, 100 and 500, between 500 and 1000, or more than 1000 subjects. Preferably, the training population includes roughly equivalent numbers of individuals that developed ovarian cancer and individuals that did not develop ovarian cancer. For purposes of characterizing the individual populations as developing or not developing ovarian cancer, any traditional method of ovarian cancer diagnosis can be used. In some embodiments, the phenotypic characteristics of the two populations used in the training set are as similar as possible but for the phenotypic characteristic of developing or not developing ovarian cancer. In another preferred embodiment, the two populations are age and postmenopausal matched.

Data for input into the mathematical models may include data representative of the measured level of biomarkers in subjects within each of the populations. Additional data for input into the mathematical models may include risk factors or factors that may lower the risk of an ovarian carcinoma such as an age greater than 55 of the woman; a family history such as women with a mother, sister, grandmother or aunt who has had ovarian cancer have a higher risk of developing the disease; genetic mutation such as women with the BRCA1 mutation have a 35 to 70 percent higher risk of ovarian cancer, and women with the BRCA2 mutation have a 10 to 30 percent higher risk; in inherited genetic disorder such as lynch syndrome or peutz-jeghers syndrome; and prior cancer such as breast, colorectal, or endometrial cancer; childbearing status such as women who have delivered at least one child, especially before age 30, are at a lower risk of developing the an ovarian carcinoma; use of birth control such as women who have used oral contraceptives for at least three months are at a lower risk of ovarian cancer; and gynecologic surgery such as a tubal ligation can reduce the risk of developing an ovarian carcinoma.

A logistic regression model uses an equation as the representation where input values (x) are combined linearly using weights or coefficient values (b) to predict an output value (y). In some embodiments, the input values (x) include the levels of the two or more biomarkers in a biological sample, and the output value (y) being modeled is binary, e.g., a differentiator between subjects that will develop ovarian cancer and subjects that will not develop ovarian cancer. The coefficient values (b) of the logistic regression algorithm may be estimated from the training data. In various embodiments, the logistic regression model may be used to test various combinations of two or more of the biomarkers to generate classifiers. The classifiers may be in the form of equations where the data representing the measured values of each of the biomarkers in the equation is multiplied by a weighted coefficient as generated by the regression model. In some embodiments, one or more weighted coefficients are a time dependent variable. For example, the training data may be grouped, classified, or identified based at least on a time dependent variable such as a time to diagnosis. The time to diagnosis variable indicates the time from when a biological sample having levels of the two or more biomarkers was obtained from the subject to the time of subject being diagnosed with an ovarian carcinoma. As such, the time to diagnosis variable allows for the classifiers to take into consideration the effect of the time to diagnosis variable on the predictive nature of the two or more biomarkers. The classifiers generated can be used to analyze input data from a test subject and provide risk assessment data.

For example, a classifier or logistic regression equation of ovarian cancer can be generated as (y)=HE4_pmol_L_+CA=−125_IU_mL+OPN_pg_mL_+Hysterectomy_Yes_No+N_preg_It6_M+Oral_Contra_Ever_Yes_No; with (b1)=550 and (b2)=*. Where (y), the dependent variable is: will develop (when y is positive) or will not develop (when y is negative) the biological feature (e.g., will or will not develop ovarian cancer). This model says that the dependent variable y depends on variables (measured values for the HE4, CA-125, and OPN; and additional data concerning whether the subject ever had a hysterectomy, became pregnant, or used an oral contraceptive) obtained from subjects in the first and second populations), plus an error term or coefficient value (b1) that encompasses a time to diagnosis of about 1.5 years (550 days), and an error term or coefficient value (b2) that encompasses various unspecified omitted factors. In some embodiments, other coefficients may be used to gauge the effect of each variable independently on the dependent variable y (e.g. a weighting factor), holding the other variables constant.

In some embodiments, the logistic regression model is fit by a maximum likelihood estimation. In other words, the coefficients are determined by maximum likelihood. A likelihood is a conditional probability. The likelihood function measures the probability of observing the particular set of variable values that occur in the sample data set. It is written as the probability of the product of the variables. The higher the likelihood function, the higher the probability of observing the variables in the sample. Maximum likelihood estimation involves finding the coefficients that makes the log of the likelihood function as large as possible or −2 times the log of the likelihood function as small as possible. In maximum likelihood estimation, some initial estimates of the coefficients are made. Then the likelihood of the data given these coefficient estimates is computed. The coefficient estimates are improved each time the data is recalculated. This process is repeated until the coefficient estimates do not change much (for example, a change of less than 0.01 or 0.001 in the probability).

In some embodiments, the classifiers or logistic regression equations are evaluated using one or more of the following methods: cross validation, Leave One out Cross Validation, n-fold cross validation, or jackknife analysis using standard statistical methods. In certain embodiments, each classifier is evaluated for its ability to properly characterize those individuals of the training population, which were not used to generate the classifier. In some embodiments, the method used to evaluate the classifier for its ability to characterize each individual of the training population is a method, which evaluates the classifiers sensitivity (TPF, true positive fraction) and 1-specificity (TNF, true negative fraction). In certain embodiments, the method used to test the classifier is Receiver Operating Characteristic (ROC), which provides several parameters to evaluate both the sensitivity and specificity of the diagnostic result of the classifier generated. The ROC area (area under the curve (AUC)) may be used to evaluate the equations. For example, an ROC area greater than 0.5, 0.6, 0.7, 0.8, 0.9 and most preferably 1.0. A perfect ROC area score of 1.0 indicates a classifier, which is both 100% sensitive and 100% specific.

### IV. Methods for Assessing Ovarian Cancer Risk

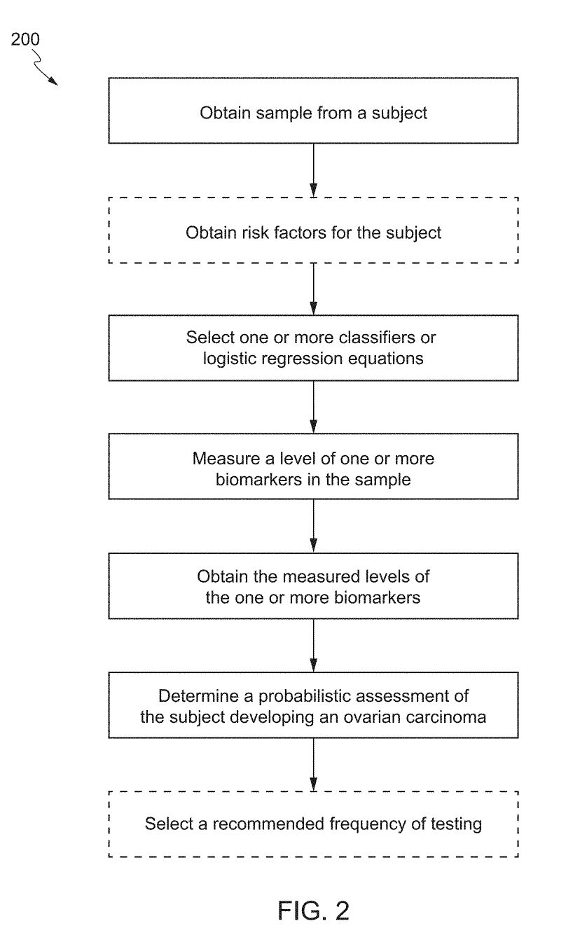

FIG. 2 depicts a simplified flowchart depicting processing performed for assessing an ovarian cancer risk according to embodiments of the present invention. The steps of FIG. 2 may be implemented in the system environment of FIG. 1, for example. As noted herein, the flowchart of FIG. 2 illustrates the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical functions. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combination of blocks in the block diagrams and/or flowchart illustration, can be implemented by special purpose hardware-based systems that perform the specified functions or acts, or combinations of special purpose hardware and computer instructions.

At step 205, a biological sample is obtained from a subject without knowledge of an ovarian mass or tumor (e.g., a preexisting ovarian mass or tumor) in the subject. In some embodiments, the subject is a patient interested in obtaining a risk assessment for ovarian cancer. For example, the subject may have one or more symptoms and/or risk factors for an ovarian carcinoma. At optional step 210, risk factors or factors that may lower the risk of an ovarian carcinoma are received or obtained at the computing system (e.g., the computing system 100 as described with respect to FIG. 1). For example, the risk factors or factors that may lower the risk of an ovarian carcinoma may be received from another computer and stored in a database with memory. In another example, the risk factors or factors that may lower the risk of an ovarian carcinoma may be received directly from the subject. In general, the risk factors or factors that may lower the risk of an ovarian carcinoma may be received from the subject or any electronic device capable of transmitting the data. The risk factors or factors that may lower the risk of an ovarian carcinoma may be received over various mediums such as wired and/or wireless mediums.

At step 215, one or more classifiers or logistic regression equations are selected for assessing the ovarian cancer risk of the subject. In various embodiments, the one or more classifiers or logistic regression equations are selected based at least on two or more biomarkers that have shown some predictive behavior for ovarian cancer. In various embodiments, the one or more classifiers or logistic regression equations are selected based at least on two or more biomarkers that have shown some predictive behavior for ovarian cancer and the benefits of the use of the two or more biomarkers at a predetermined time prior to diagnosis of an ovarian carcinoma. Optionally, the risk factors or factors that may lower the risk of an ovarian carcinoma of the subject may also be used to select the one or more classifiers or logistic regression equations. For example, the one or more classifiers or logistic regression equations may be selected based on at least one of: (i) the two or more biomarkers, (ii) benefits of the use of the two or more biomarkers at a predetermined time prior to diagnosis, and (iii) the risk factors or factors that may lower the risk of an ovarian carcinoma risk factors.

At step 220, the levels of two or more biomarkers in the biological sample are measured. In various embodiments, the levels of the two or more biomarkers are measured using any kit or analytical test known for quantitatively measuring the two or more biomarkers in the sample. In various embodiments, the two or more biomarkers to be measured are the same two or more biomarkers used in step 215 to select the one or more classifiers or logistic regression equations. In some embodiments, the two or more biomarkers to be measured are selected based on the two or more classifiers or logistic regression equations selected for assessing the ovarian cancer risk of the subject. At step 225, the measured levels of the two or more biomarkers are received or obtained at the computing system (e.g., the computing system 100 as described with respect to FIG. 1). For example, the measured levels may be received from another computer and stored in a database with memory. In another example, the measured levels may be received directly from an analytical device. In general, the measured levels may be received from any electronic device capable of transmitting analytical data. The measured levels may be received over various mediums such as wired and/or wireless mediums.

At step 230, a probabilistic assessment of the subject developing an ovarian carcinoma is determined. In various embodiments, assessing the cancer risk includes the execution of the one or more classifiers or logistic regression equations using the measured levels of the two or more biomarkers and optionally the risk factors or factors that may lower the risk of an ovarian carcinoma. The one or more classifiers or logistic regression equations may be developed as discussed herein using training data from subjects in various populations. In one example, the one or more classifiers or logistic regression equations includes two or more biomarkers computed by analyzing prior diagnostic data and personal risk factors. The one or more classifiers or logistic regression equations may be adjusted for factors such as menopause, age, time to diagnosis, etc. Thereafter, the probabilistic assessment of developing an ovarian carcinoma is developed through execution of the one or more classifiers or logistic regression equations. For example, the one or more classifiers or logistic regression equations expresses the log-odds (natural log of the probabilistic ratio of an event occurring versus the event not occurring) for cancer as a combination of the biomarkers and any weights or coefficients such as time to diagnosis. Alternatively, the one or more classifiers or logistic regression equations expresses the log-odds (natural log of the probabilistic ratio of an event occurring versus the event not occurring) for cancer as a combination of the biomarkers, the risk factors or factors that may lower the risk of an ovarian carcinoma, and any weights or coefficients such as time to diagnosis. Determination of significant predictors may employ the log-likelihood ratio test, which is a statistical test for making a decision between two hypotheses based on the value of this ratio. The maximum likelihood estimates for the logistic regression weights or coefficients may be obtained for each biomarker and optionally each risk factor, and the corresponding odds ratios are estimated with associated confidence intervals. The log-odds ratio provided by the model is converted into a corresponding probability of developing ovarian cancer. Thus, the system utilizes the information obtained from the biomarker analysis as well as the person-specific risk factors and any other variables such as time to diagnosis to perform a logistic regression thus producing a probabilistic assessment of the person developing ovarian cancer.

At optional step 235, a recommended frequency of follow-up testing for the two or more biomarkers for effective risk assessment is selected. In various embodiments, the recommended frequency of follow-up testing is selected based on the one or more classifiers or logistic regression equations selected to determine the probabilistic assessment of the subject.

### V. Examples

Without intending to limit the scope of the embodiments discussed herein, the systems and methods implemented in various embodiments may be better understood by referring to the following examples.

The goal of the following examples were to examine the utility of various algorithms in their ability to determine the risk of a post-menopausal woman developing an ovarian carcinoma using specimens collected prior to diagnosis of known ovarian cancer patients (cases) and matched healthy controls (controls). Further, the clinical utility of the various algorithms were evaluated with specimens collected at increasing intervals preceding the time of diagnosis, such that the recommended frequency of testing can be established for effective risk prediction.

### Assay Description

Six (6) protein biomarkers were measured in serum. The six biomarkers were HE4, CA-125, Leptin, OPN, Prolactin, and IGF2. The six biomarker measurements were made using a common aliquot for each specimen tested on three independent assays per their respective standard operating procedures (SOPs). Firstly, Leptin, OPN, and Prolactin were measured on a multiplexed sandwich ELISA (enzyme-linked immunosorbent assay) co-developed by LabCorp and Aushon Biosystems (Billerica, Mass.). Total IGF2 was measured on a sandwich ELISA developed by Mediagnost (Reutlingen, Germany), which includes an acid dissociation and, subsequent, IGF1 blocking step to ameliorate interference from IGF binding proteins. For the measurement of HE4 and CA125 in serum, an aliquot of serum was mixed with an equal volume of third-party diluent and then both analytes were measured from the common diluted specimen on the FDA-approved Elecsys assays using the COBAS e602 system without further modification. Analytical performance characteristics of the assays for the six biomarkers have been established through independent validation studies conducted previously by LabCorp.

### Samples and Study Design

Retrospective serum specimens were obtained, which were originally procured during an Ovarian Cancer Screening study. All specimens were obtained from the “control” arm of the study, in which post-menopausal women, 50 to 74 years of age were unscreened for the presence of ovarian cancer by either ultrasound or other diagnostic tests (e.g., CA-125). Women at high risk for development of ovarian cancer were excluded from the trial and, thus, from these studies based on: (1) a history of bilateral oophorectomy, (2) active non-ovarian cancer malignancy (women with a past history of malignancy were eligible if they had no documented persistent or recurrent disease), (3) a previous history of ovarian cancer malignancy, or (4) a familial/genetic predisposition for development of ovarian cancer. All diagnoses of ovarian cancer were confirmed by the independent committee of the study.

A cohort of case/control studies was collected at time points before the diagnosis of ovarian cancer in order to assess the predictive utility of a protein biomarker panel and associated algorithm at predetermined times prior to diagnosis, and to establish the necessary frequency for testing. The cohort of case/control studies served as a training cohort by which the predictive algorithm(s) were initially developed.

### Training

The specimens for the training cohort were identified and comprised 100 cases and 100 controls, each with only a single specimen. The specimens from the 100 cases are characterized by their time of collection in relation to the time of ovarian cancer diagnosis.

### Cases:

The cases were from women volunteers diagnosed with primary invasive epithelial ovarian/fallopian or primary peritoneal cancer (borderline and non-epithelial cancers were excluded) up to 3 years following specimen collection, which was collected at the time of recruitment in the study. A breakdown with respect to the diagnosis and time to diagnosis relative to the specimen collection are shown in Tables 1 and 2.

TABLE 1

| Diagnosis | Count |
|---|---|
| primary invasive epithelial ovarian cancers (ICD10 C56) | 94 |
| primary invasive epithelial tubal cancers (ICD10 C57.0) | 4 |
| primary peritoneal cancers (ICD10 C48) | 2 |

TABLE 2

| Specimen Collection in Relation to Diagnosis | Count |
|---|---|
| up to 12 months prior to diagnosis | 26 |
| >12 and up to 24 months prior to diagnosis | 30 |
| >24 and up to 36 months prior to diagnosis | 44 |

### Controls:

The controls were from healthy women volunteers with no cancer recorded at the time of specimen selection in 2013. The healthy controls did include those women who, after the date the sample was collected, may have had a non-cancer related record in the Hospital Episode Statistics Database or a record for a benign condition in the Cancer Registry Database or are now deceased (due to a non-cancer ICD10 code). Each control sample was randomly matched to each of the cases following the order of the following criteria: The sample from the healthy control is from the same center as the case; The age of the healthy control volunteer at the time of sample collection (recruitment) is within +/−2 years of the age of the ovarian cancer case volunteer at the time of sample collection (recruitment); The date of sample collection for the healthy control volunteer is within +/−2 years of the date of sample collection for the ovarian cancer case volunteer; and the time-to-spin for the sample collected from the healthy control volunteer is within +/−2 hours of the time-to-spin for the sample collected from the ovarian case volunteer.

### Cancer Assessment Model Construction

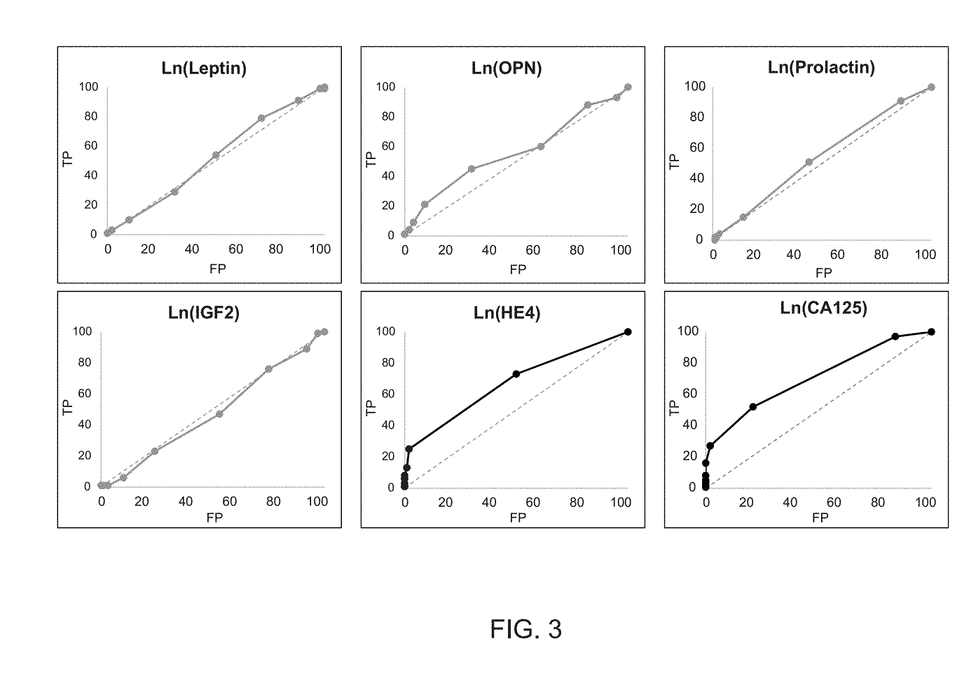

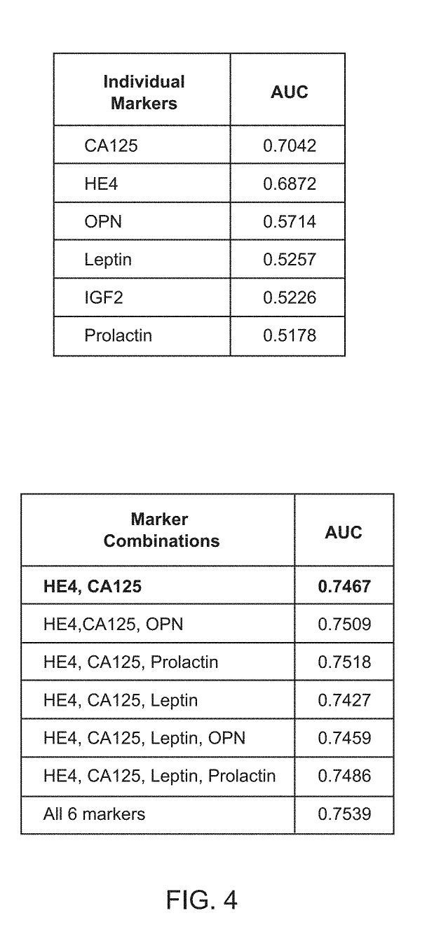

Logistic regression equations were generated to evaluate the effectiveness of using each of the six biomarkers: HE4, CA-125, Leptin, OPN, Prolactin, and IGF2 independently to predict diagnosis of ovarian cancer in the 100 cases/100 controls. As shown in FIG. 3, ROC curves were plotted for the logistic regression analysis of each of the six biomarkers. The ROC curves provide various information, for example, they show the tradeoff between sensitivity and specificity (any increase in sensitivity will be accompanied by a decrease in specificity), closer the curve follows the left-hand border and then the top border of the ROC space, the more accurate the test, looser the curve comes to the 45-degree diagonal of the ROC space, the less accurate the test, slope of the tangent line at a cutpoint gives the likelihood ratio (LR) for that value of the test, and area under the curve (AUC) is a measure of test accuracy (see, e.g., FIG. 4). Qualitative review of the ROC curves indicated the greatest potential utility in the biomarkers CA-125 and HE4 based on the aforementioned information provided by the curves and AUC.

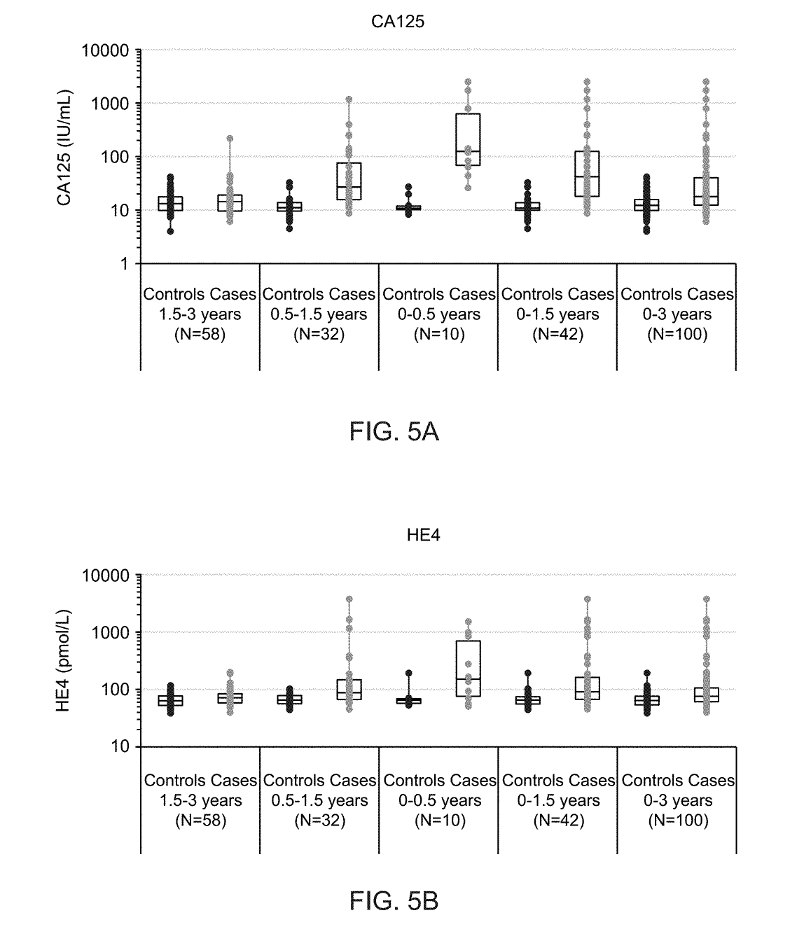

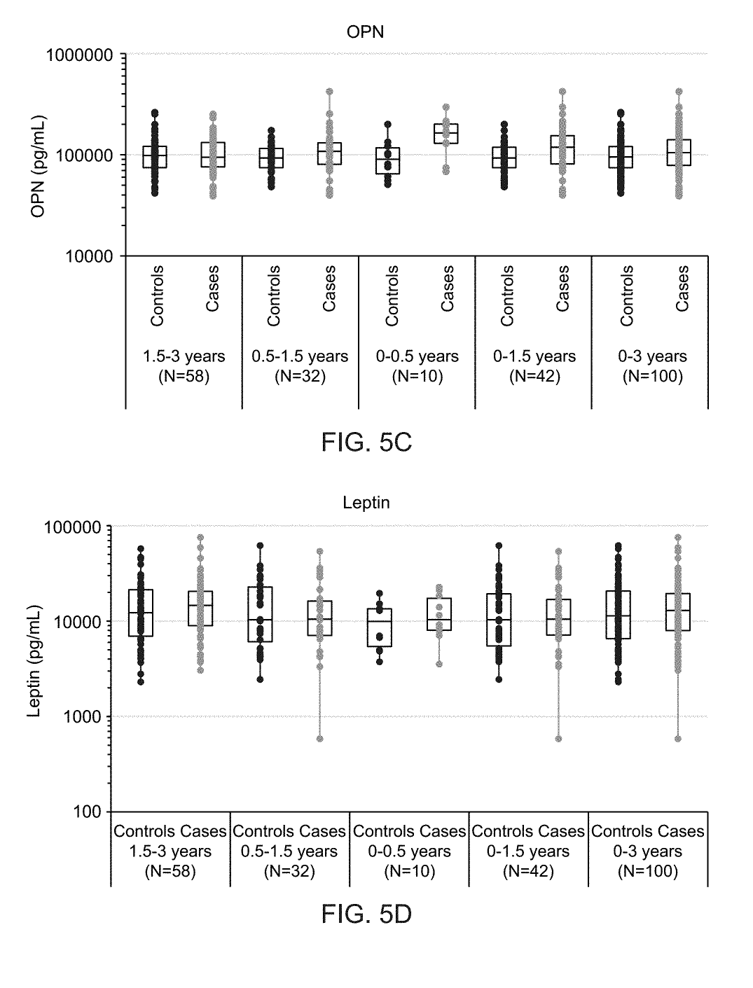

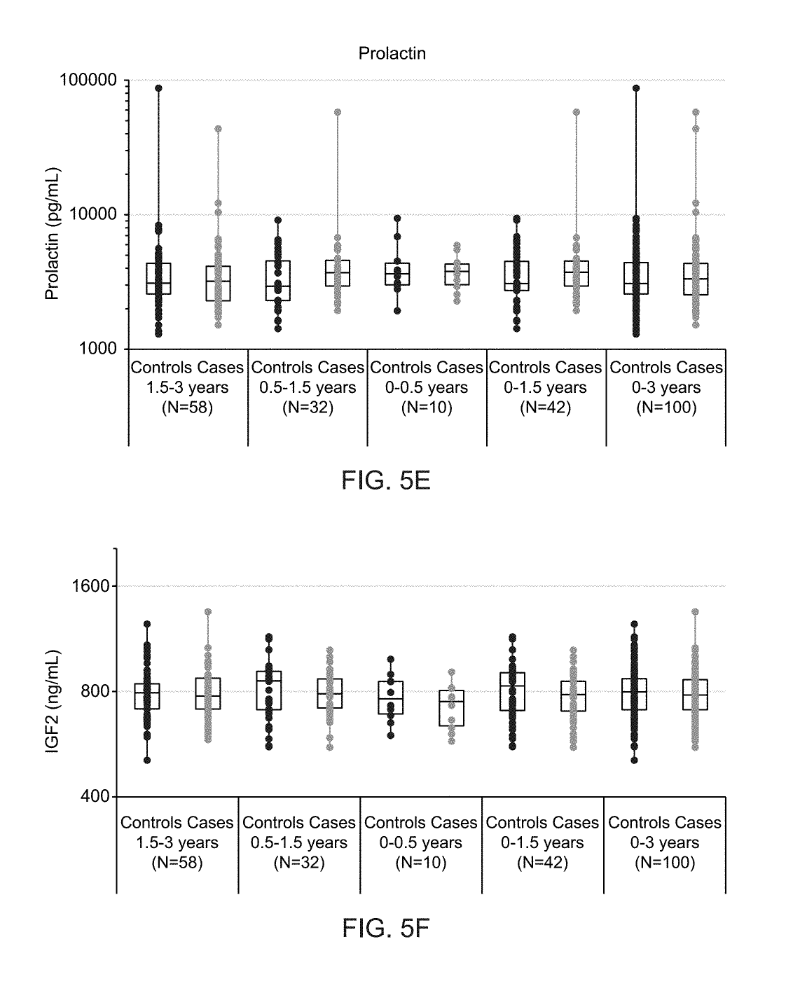

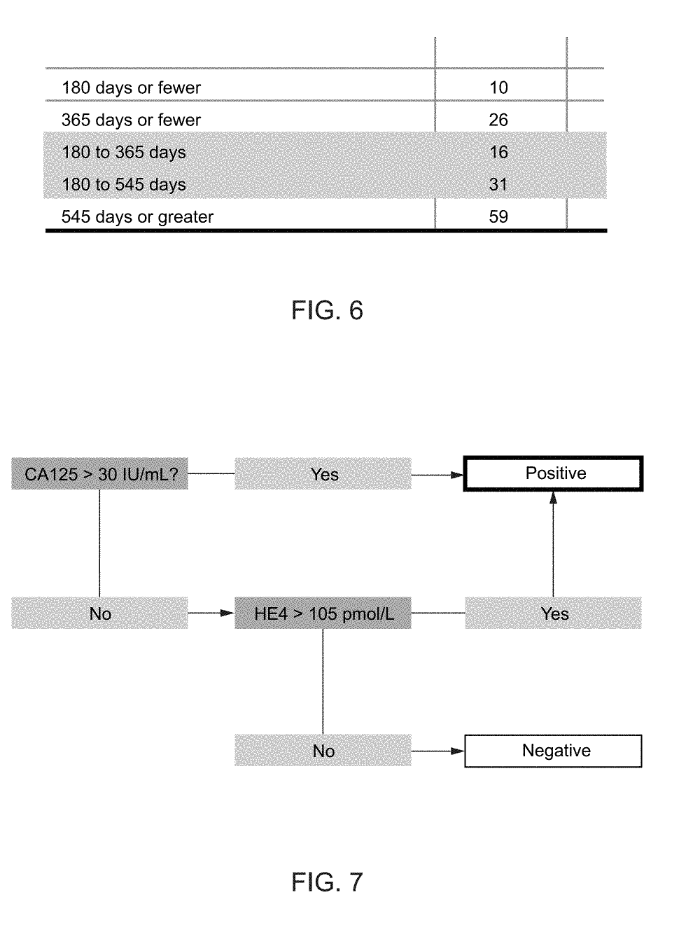

In order to determine a risk of a subject to develop ovarian cancer, mathematical models as described in detail herein were used to generate “classifiers” which differentiate between subjects that will develop ovarian cancer (Cases) and subjects that will not develop ovarian cancer (Controls). As shown in FIGS. 5A-5F, CA-125 and HE4 showed increasing differentiation (i.e., separation) with reduction in time to diagnosis, while OPN only showed differentiation with time to diagnosis of less than six months or less. Leptin Prolactin, and IGF2 showed no differentiation regardless of the time to diagnosis. In particular, it was demonstrated that there is potential clinical application for mathematical models to assess the risk of a subject developing an ovarian carcinoma when the time to diagnosis is between 0.5 and 1.5 years using CA-125 and HE4 (see, e.g., the accumulated data shown in FIG. 6). As such, a decision tree algorithm was developed to assess the risk of a subject developing an ovarian carcinoma using the CA-125 and HE4, as shown in FIG. 7. Moreover, because it was determined from the mathematical models that CA-125 and HE4 are particularly effective in predictions made for subjects that are 0.5 to 1.5 years from diagnosis, a recommendation can be made to the subject on the frequency of follow-up testing, e.g., every 6 months.

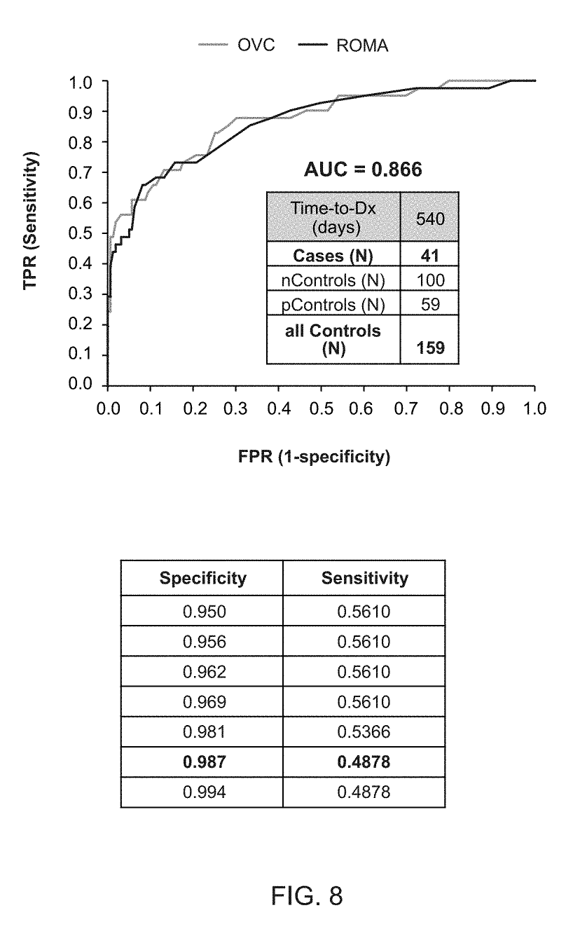

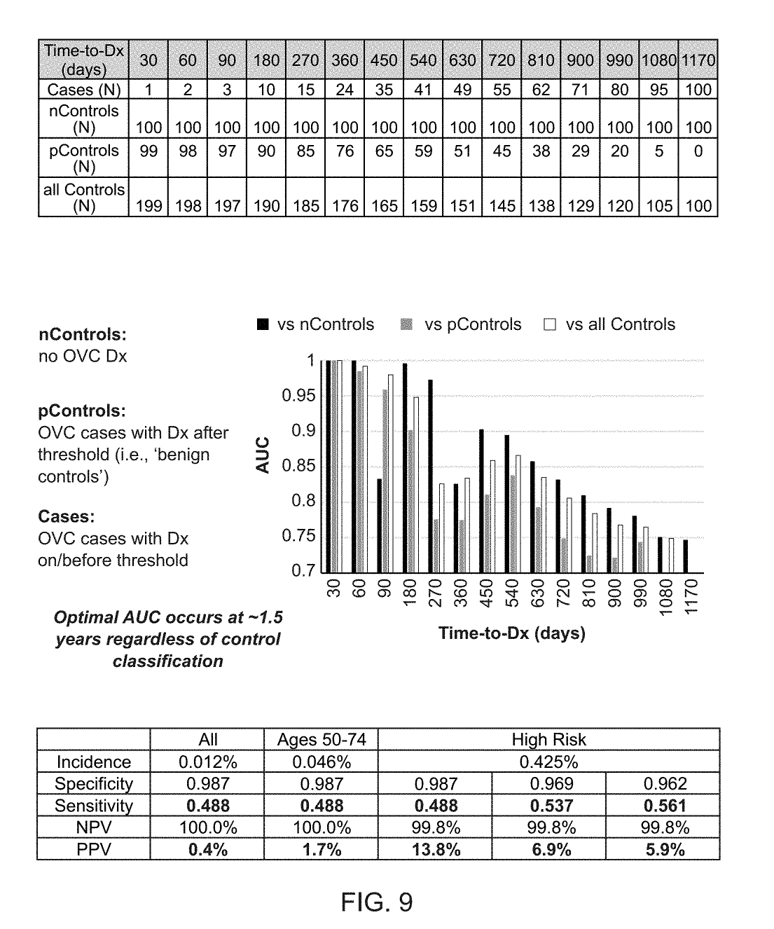

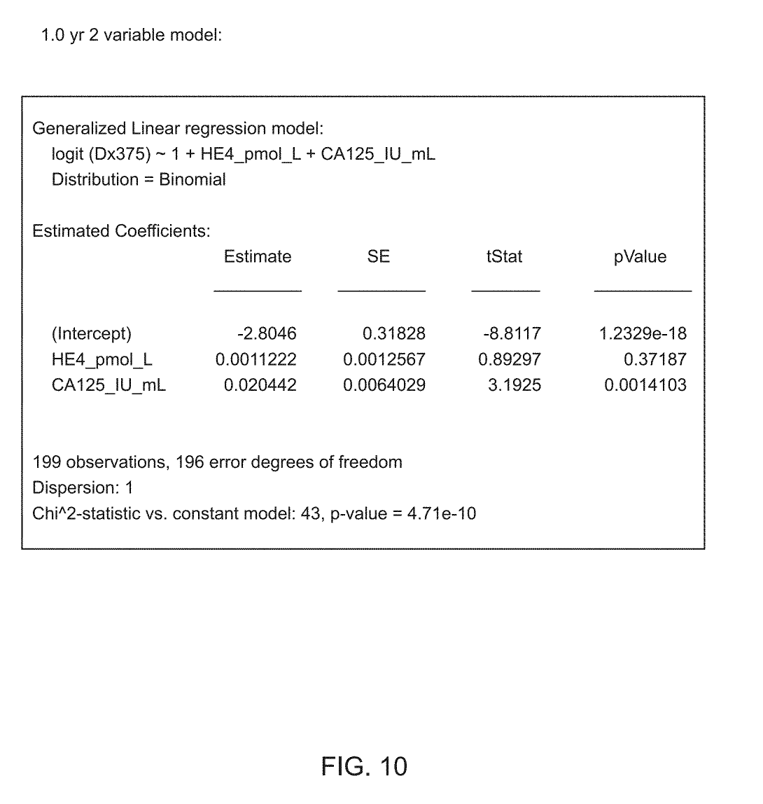

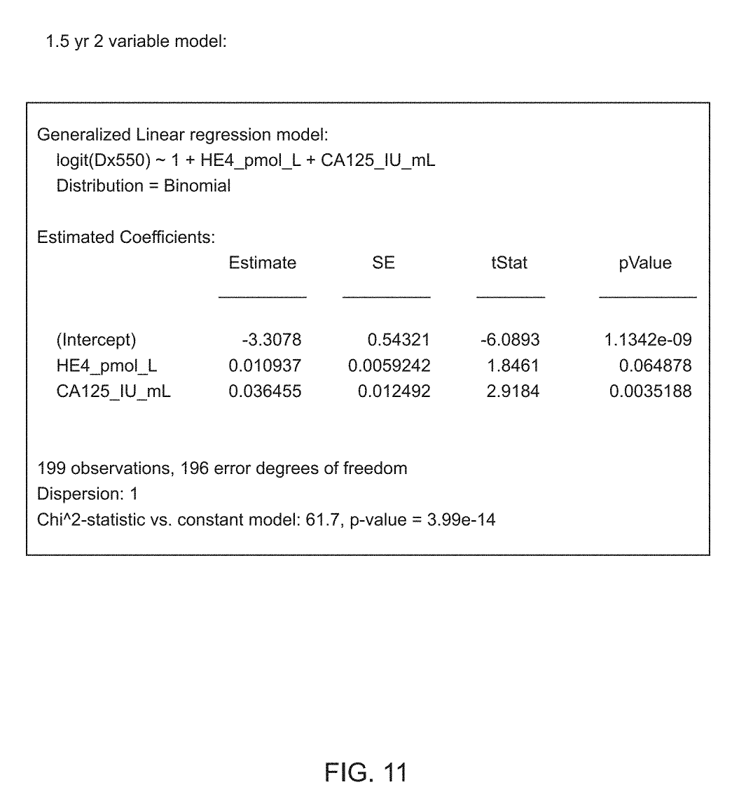

In order to enhance the determination of a risk of a subject to develop ovarian cancer, mathematical models as described in detail herein were used to generate “classifiers” which differentiate between subjects that will develop ovarian cancer (Cases) and subjects that will not develop ovarian cancer (Controls) using various combinations of biomarkers and additional data. For example, the mathematical models may be trained with a training cohort grouped, classified, or identified based at least on a time dependent variable such as a time to diagnosis. Thereafter, the logistic regression equations could take as input any number of variables (e.g., HE4 values and CA-125 values) to enhance the determination of a risk of a subject to develop ovarian cancer. As shown in FIGS. 8 and 9, the time to diagnosis variable allows the classifiers to take into consideration the effect of the time to diagnosis variable on the predictive nature of the two or more biomarkers. Specifically, FIGS. 8 and 9 show the AUC of the ROC for an exemplary logistic regression equation optimized with the time to diagnosis of 1.5 years to diagnosis. Thus, it was demonstrated that the use of biomarker and time to diagnosis variables with logistic regression equations add value to the determination of a risk of a subject to develop ovarian cancer. FIGS. 10 and 11 show examples of two variable models for 1.0 years and 1.5 years from time of diagnosis that take into consideration the effect of the time to diagnosis variable on the predictive nature of the two or more biomarkers.

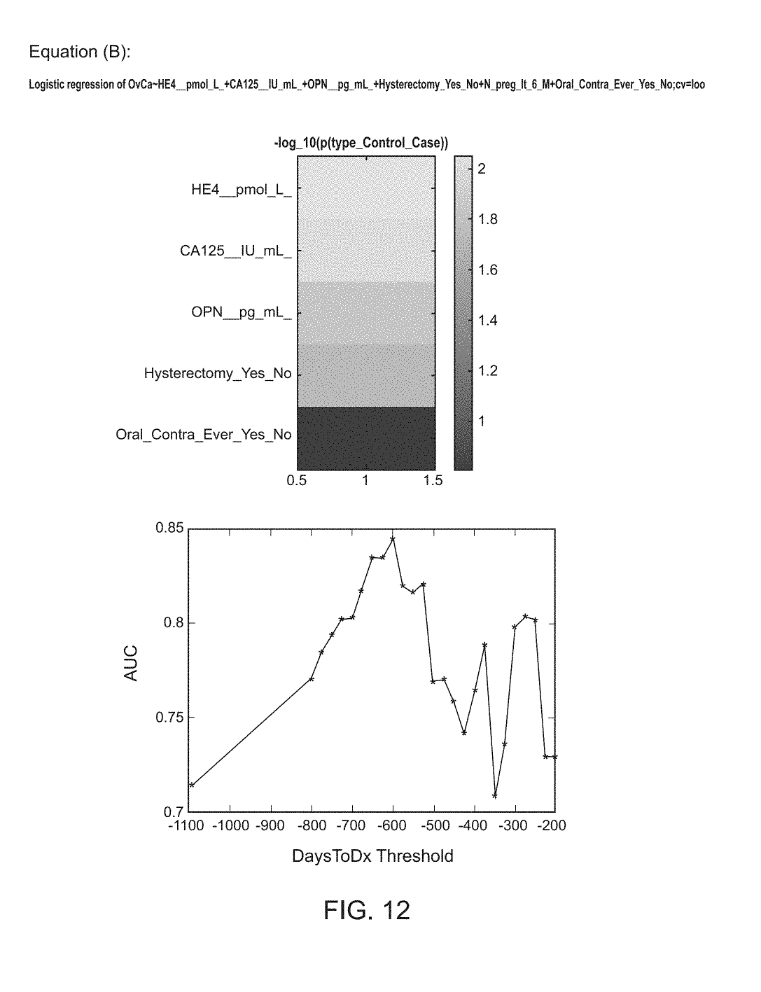

In order to further enhance the determination of a risk of a subject to develop ovarian cancer, mathematical models as described in detail herein were used to generate “classifiers” which differentiate between subjects that will develop ovarian cancer (Cases) and subjects that will not develop ovarian cancer (Controls) using various combinations of biomarkers and additional data. For example, the mathematical models may be trained with a training cohort grouped, classified, or identified based at least on a time dependent variable such as a time to diagnosis. Thereafter, the logistic regression equations could take as input any number of variables (e.g., HE4 values, CA-125 values, OPN values, and risk factors or factors that may lower the risk of an ovarian carcinoma) to enhance the determination of a risk of a subject to develop ovarian cancer. FIG. 12 shows the UAC of the ROC for an exemplary logistic regression equation (A) taking HE4 values, CA-125 values, OPN values, risk factors or factors that may lower the risk of an ovarian carcinoma (hysterectomy, pregnancy, and contraception) and being maximized with the time to diagnosis of ˜600 days. Thus, the use of additional variables with logistic regression equations adds some value to the determination of a risk of a subject to develop ovarian cancer.

### Model Validation

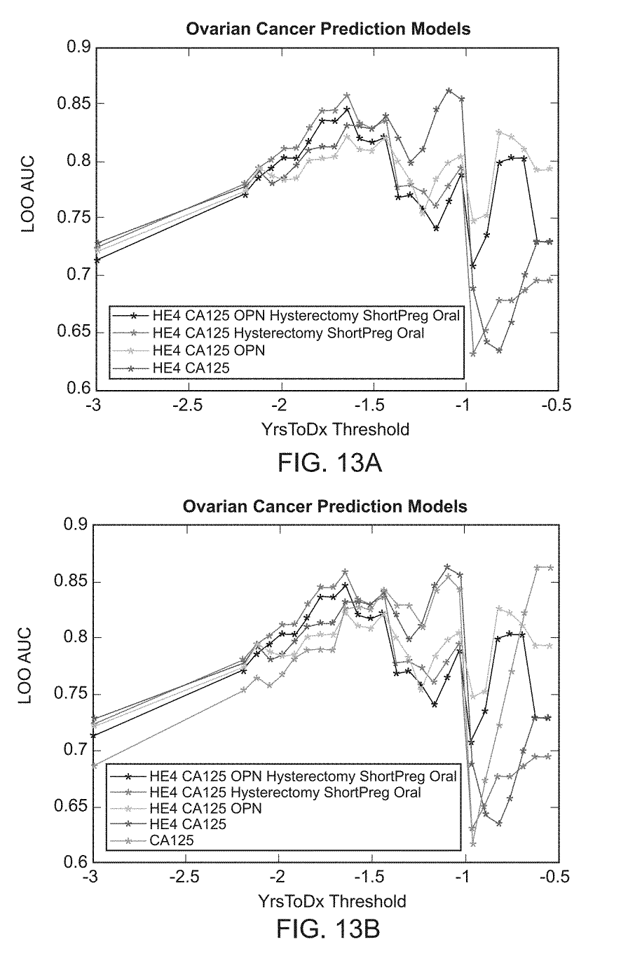

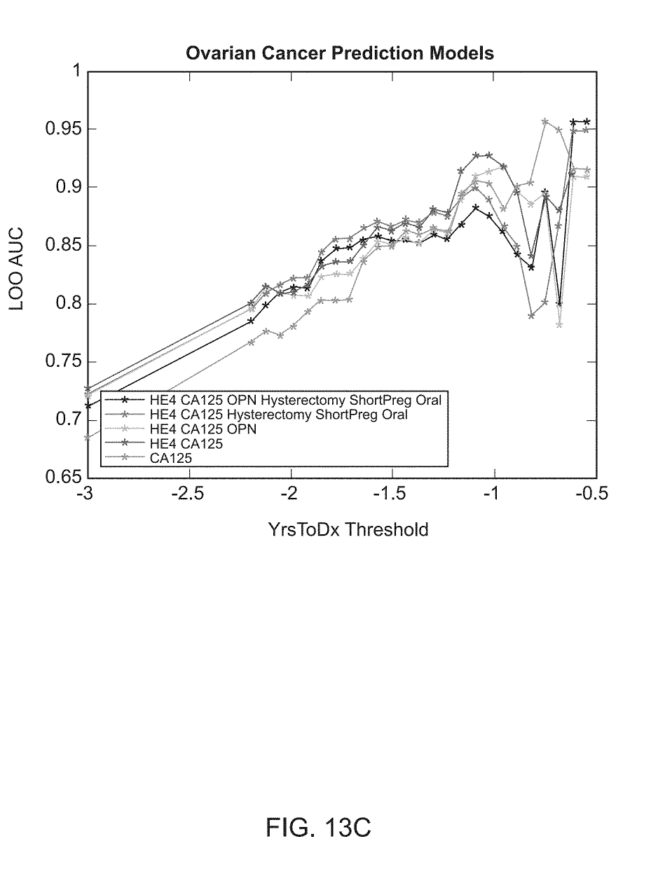

The indication modeling is an iterative process that includes validation to ensure the modeling accurately predicts the risk of developing ovarian cancer. A statistical approach was taken to validate the model and verify risk assessment from the model using training cohorts. Validation cohorts served to independently validate the clinical sensitivity and specificity of the algorithm(s) generated from the training cohort and, were further structured to inform on the recommended frequency of testing. FIGS. 13A and 13B show cross-validated time-thresholded AUCs for different models used to generate classifiers or logistic regression equations. FIG. 13C shows cross-validated time-thresholded AUCs for different models (excluding positives>time threshold rather than treating them as negatives) used to generate classifiers or logistic regression equations.

While the invention has been described in detail, modifications within the spirit and scope of the invention will be readily apparent to the skilled artisan. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by the skilled artisan. Furthermore, the skilled artisan will appreciate that the foregoing description is by way of example only, and is not intended to limit the invention.

What is claimed is:

1. A system comprising:
a logistic regression model that uses an equation comprising:
input values comprising measured levels of two or more biomarkers in a sample obtained from a subject without knowledge of an ovarian mass or tumor in the subject, wherein the two or more biomarkers include Human epididymal protein 4 (HE4) and Cancer Antigen 125 (CA-125); and
coefficient values that take into consideration a time to diagnosis variable,
wherein the input values are combined linearly using the coefficient values to predict an output value, and the output value is a differentiator between subjects that will develop ovarian cancer and subjects that will not develop ovarian cancer;
one or more processors and non-transitory machine readable storage medium; and
program instructions to determine a probabilistic assessment of the subject developing an ovarian carcinoma based at least on the logistic regression model,
wherein the program instructions are stored on the non-transitory machine readable storage medium for execution by the one or more processors.

2. The system of claim 1, wherein the time to diagnosis variable is estimated from training data and indicates a time from when biological samples having levels of the two or more biomarkers were obtained from test subjects to a time of the test subjects, respectively, being diagnosed with the ovarian carcinoma.

3. The system of claim 2, wherein the determining the probabilistic assessment comprises executing the equation using the measured levels of the two or more biomarkers and the coefficient values.

4. The system of claim 3, wherein the input values further comprise risk factors or factors that lower the risk of the ovarian carcinoma.

5. The system of claim 4, wherein the risk factors include at least one of the following: age of the subject, family history, genetic mutation, inherited genetic disorder, and prior cancer.

6. The system of claim 5, wherein the factors that lower the risk of the ovarian carcinoma include at least one of the following: child bearing status, use of birth control, use of oral contraceptives, and prior gynecological surgery.

7. The system of claim 3, wherein the two or more biomarkers further include one or more biomarkers that has not been demonstrated previously to be predictive for the development ovarian cancer.

8. A non-transitory machine readable storage medium having instructions stored thereon that when executed by one or more processors cause the one or more processors to perform a method comprising:
selecting a logistic regression model, wherein the logistic regression model uses an equation comprising: (i) input values comprising measured levels of two or more biomarkers in a sample obtained from a subject without knowledge of an ovarian mass or tumor in the subject; and (ii) coefficient values estimated from training data that take into consideration various unspecified omitted factors, wherein the input values are combined linearly using the coefficient values to predict an output value, and the output value is a differentiator between subjects that will develop ovarian cancer and subjects that will not develop ovarian cancer; and
determining a probabilistic assessment of the subject developing ovarian cancer based at least on the logistic regression model.

9. The non-transitory machine readable storage medium of claim 8, wherein the two or more biomarkers include two or more biomarkers that have not been demonstrated previously to be predictive for the development of ovarian cancer.

10. The non-transitory machine readable storage medium of claim 8, wherein the two or more biomarkers include any combination of: Human epididymal protein 4 (HE4), Cancer Antigen 125 (CA-125), Leptin, Osteopontin (OPN), Prolactin, and Insulin-like Growth Factor 2 (IGF2).

11. The non-transitory machine readable storage medium of claim 8, wherein the equation further comprise: (iii) coefficient values estimated from training data that take into consideration a time to diagnosis variable, wherein the time to diagnosis variable indicates a time from when biological samples having levels of the two or more biomarkers were obtained from test subjects to a time of the test subjects, respectively, being diagnosed with the ovarian carcinoma.

12. The non-transitory machine readable storage medium of claim 8, wherein the determining the probabilistic assessment comprises executing the equation using the measured levels of the two or more biomarkers and the coefficient values.

13. The non-transitory machine readable storage medium of claim 8, wherein the input values further comprise risk factors or factors that lower the risk of the ovarian carcinoma.

14. The non-transitory machine readable storage medium of claim 8, wherein the method further comprises storing the probabilistic assessment.

15. A method comprising:
selecting, using a computing device, a logistic regression model, wherein the logistic regression model uses an equation comprising: (i) input values comprising measured levels of two or more biomarkers in a sample obtained from a subject without knowledge of an ovarian mass or tumor in the subject; and (ii) coefficient values estimated from training data that take into consideration various unspecified omitted factors, wherein the input values are combined linearly using the coefficient values to predict an output value, and the output value is a differentiator between subjects that will develop ovarian cancer and subjects that will not develop ovarian cancer; and
determining, using the computing device, a probabilistic assessment of the subject developing ovarian cancer based at least on the logistic regression model.

16. The method of claim 15, wherein the two or more biomarkers include two or more biomarkers that have not been demonstrated previously to be predictive for the development of ovarian cancer.

17. The method of claim 15, wherein the two or more biomarkers include any combination of: Human epididymal protein 4 (HE4), Cancer Antigen 125 (CA-125), Leptin, Osteopontin (OPN), Prolactin, and Insulin-like Growth Factor 2 (IGF2).

18. The method of claim 15, wherein the equation further comprise: (iii) coefficient values estimated from training data that take into consideration a time to diagnosis variable, wherein the time to diagnosis variable indicates a time from when biological samples having levels of the two or more biomarkers were obtained from test subjects to a time of the test subjects, respectively, being diagnosed with the ovarian carcinoma.

19. The method of claim 15, wherein the determining the probabilistic assessment comprises executing the equation using the measured levels of the two or more biomarkers and the coefficient values.

20. The method of claim 15, wherein the input values further comprise risk factors or factors that lower the risk of the ovarian carcinoma.

21. A method for assessing risk of developing ovarian cancer, the method comprising:
obtaining, by a computing device, measured levels of two or more biomarkers in a sample obtained from a subject without knowledge of an ovarian mass or tumor in the subject, wherein the two or more biomarkers include Human epididymal protein 4 (HE4) and Cancer Antigen 125 (CA-125);
determining, by the computing device, a probabilistic assessment of the subject developing ovarian cancer based at least on the obtained values and coefficient values estimated from training data that take into consideration a time to diagnosis variable; and
storing, by the computing device, the probabilistic assessment.

22. The method of claim 21, further comprising selecting, by the computing device, one or more classifiers or logistic regression equations for assessing the risk of the subject developing ovarian cancer.

23. The method of claim 22, wherein the determining the probabilistic assessment comprises executing the one or more classifiers or logistic regression equations using the measured levels of the two or more biomarkers and the coefficient values.

24. The method of claim 23, further comprising obtaining, by the computing system, one or more risk factors or factors that lower the risk of the ovarian carcinoma.

25. The method of claim 24, wherein the one or more classifiers or logistic regression equations are selected based on at least one of: (i) the two or more biomarkers, and (ii) the risk factors or factors that lower the risk of the ovarian carcinoma.

26. The method of claim 25, wherein the determining the probabilistic assessment comprises executing the one or more classifiers or logistic regression equations using the measured levels of the two or more biomarkers and the one or more risk factors or factors that lower the risk of the ovarian carcinoma.

27. The method of claim 26, further comprising providing, by the computing system, a recommended frequency of follow-up testing for the two or more biomarkers based on the one or more classifiers or logistic regression equations selected to determine the probabilistic assessment of the subject.

28. A non-transitory machine readable storage medium having instructions stored thereon that when executed by one or more processors cause the one or more processors to perform a method comprising:
obtaining measured levels of two or more biomarkers in a sample obtained from a subject without knowledge of an ovarian mass or tumor in the subject, wherein the two or more biomarkers include Human epididymal protein 4 (HE4) and Cancer Antigen 125 (CA-125);
determining a probabilistic assessment of the subject developing ovarian cancer based at least on the obtained values and coefficient values estimated from training data that take into consideration a time to diagnosis variable; and
storing the probabilistic assessment.

29. The non-transitory machine readable storage medium of claim 28, wherein the method further comprises selecting one or more classifiers or logistic regression equations for assessing the risk of the subject developing ovarian cancer.

30. The non-transitory machine readable storage medium of claim 29, wherein the determining the probabilistic assessment comprises executing the one or more classifiers or logistic regression equations using the measured levels of the two or more biomarkers.

31. The non-transitory machine readable storage medium of claim 30, wherein the method further comprises obtaining one or more risk factors or factors that lower the risk of the ovarian carcinoma.

32. The non-transitory machine readable storage medium of claim 31, wherein the one or more classifiers or logistic regression equations are selected based on at least one of: (i) the two or more biomarkers, and (ii) the risk factors or factors that lower the risk of the ovarian carcinoma.

33. The non-transitory machine readable storage medium of claim 32, wherein the determining the probabilistic assessment comprises executing the one or more classifiers or logistic regression equations using the measured levels of the two or more biomarkers and the risk factors or factors that lower the risk of the ovarian carcinoma.

34. The non-transitory machine readable storage medium of claim 33, wherein method further comprises providing a recommended frequency of follow-up testing for the two or more biomarkers based on the one or more classifiers or logistic regression equations selected to determine the probabilistic assessment of the subject.

35. A system comprising:
one or more processors and non-transitory machine readable storage medium;
program instructions to obtain, by a computing device, measured levels of two or more biomarkers in a sample obtained from a subject without knowledge of an ovarian mass or tumor in the subject, wherein the two or more biomarkers include Human epididymal protein 4 (HE4) and Cancer Antigen 125 (CA-125);
program instructions to determine, by the computing device, a probabilistic assessment of the subject developing ovarian cancer based at least on the obtained values and coefficient values estimated from training data that take into consideration a time to diagnosis variable; and
program instructions to store, by the computing device, the probabilistic assessment,
wherein the program instructions are stored on the non-transitory machine readable storage medium for execution by the one or more processors.

36. The system of claim 35, further comprising program instructions to select, by the computing device, one or more classifiers or logistic regression equations for assessing the risk of the subject developing ovarian cancer, wherein the determining the probabilistic assessment comprises executing the one or more classifiers or logistic regression equations using the measured levels of the two or more biomarkers.

37. The system of claim 36, further comprising program instructions to obtain, by the computing system, one or more risk factors or factors that lower the risk of the ovarian carcinoma.

38. The system of claim 37, wherein the one or more classifiers or logistic regression equations are selected based on at least one of: (i) the two or more biomarkers, and (ii) the risk factors or factors that lower the risk of the ovarian carcinoma.

39. The system of claim 38, wherein the determining the probabilistic assessment comprises executing the one or more classifiers or logistic regression equations using the measured levels of the two or more biomarkers and the one or more risk factors or factors that lower the risk of the ovarian carcinoma.

40. The system of claim 35, further comprising program instructions to provide, by the computing system, a recommended frequency of follow-up testing for the two or more biomarkers based on the one or more classifiers or logistic regression equations selected to determine the probabilistic assessment of the subject.
